(19) United States
(12) Patent Application Publication
(10) Pub. No.: US 2021/0283421 A1
(43) Pub. Date: Sep. 16, 2021

(54) ULTRA-COMPACT PHOTOTHERAPY APPARATUS FOR TREATING SKIN DISEASE AND HEALING WOUNDS

(71) Applicant: EVORAY Co., Ltd., Cheongju-si (KR)

(72) Inventors: Won Gyu KANG, Cheongju-si (KR); Pok Hyeon JEON, Cheongju-si (KR); Jung Ho CHOI, Cheongju-si (KR)

(73) Assignee: EVORAY Co., Ltd., Cheongju-si (KR)

(21) Appl. No.: 17/033,977

(22) Filed: Sep. 28, 2020

(30) Foreign Application Priority Data
Mar. 11, 2020 (KR) 10-2020-0030262

(51) Int. Cl. A61N 5/06; H05K 1/18; H01M 2/10

(52) U.S. Cl. CPC A61N 5/0616; H05K 1/182; H01M 2/1044; H05K 2201/10037; H05K 2201/10106; A61N 2005/067; A61N 2005/0651; A61N 2005/0661; A61N 2005/0663; A61N 2005/066; H01M 2220/30

(57) ABSTRACT

An ultra-compact phototherapy apparatus for treating skin diseases includes: a PCB substrate in which a light emitting element and a pair of left and right first metal electrodes (positive pole) are formed on an upper surface and a second metal electrode is formed on a lower surface, a battery having a first electrode on an upper surface, which is in contact with the second metal electrode on the lower surface of the PCB substrate, and a second electrode on a lower surface, and an electrode body formed so that the battery is inserted inside and the PCB substrate is inserted in slidingly a front-rear direction, of which an upper inner side is selectively in contact with the first metal electrode of the PCB substrate.

### CROSS-REFERENCE TO RELATED APPLICATIONS

This application claims priority to and the benefit of Korean Patent Application No. 10-2020-0030262, filed on Mar. 11, 2020, the disclosure of which is incorporated herein by reference in its entirety.

### TECHNICAL FIELD

The present invention relates to a phototherapy apparatus for a wearable based on a light emitting diode (LED), and more specifically, to a patch-type ultra-compact phototherapy apparatus for treating skin diseases, which is used by adhering to skin to heal a wound site quickly and effectively by improving cell migration and proliferation, and to relieve pain.

### BACKGROUND

Phototherapy is a treatment that stimulates the biochemical reaction of a human body by exposing it to light, and is widely used to heal and relieve wounds using medical LED devices or laser devices.

However, conventional medical LED devices or laser devices have problems that are not only less portable, but also inflexible, difficult to uniformly irradiate light over a wide area, and generate excessive heat, and thus there was a limit that they could not be in close contact with the human body in order to enhance the therapeutic effect.

In order to solve the problems, recently, a patch-type phototherapy apparatus has been developed, which may sustain highly efficient treatment while allowing a daily life regardless of time and place by attaching a light and flexible band-shaped light source to the skin like an adhesive plaster.

All of an OLED, a battery, an overheat protection device, and a patch, which are the main components of the apparatus, have a form of a thin film, a thickness of less than 1 mm, a weight of less than 1 gram, and operate for a long time of 300 hours or more, and may be driven even when bent within a radius of 20 mm, and a shape and size of the patch may be adjusted variously, so that they may be conveniently attached to the human body.

In addition, since light may be emitted in a flexible form over the entire surface, the uniformity of light is guaranteed, and there is an advantage that the required amount of light may be reached with relatively small power.

Meanwhile, as an example of the patch-type phototherapy apparatus, Patent Document 1 discloses “a skin-adhesive light emitting diode patch assembly provided with an insoluble hydrogel layer for maintaining irradiation intensity”.

As shown in FIG. 1, the patch assembly is configured to include a circuit board 110 on which one or more light emitting diodes 120 are mounted, a battery 150 that is electrically connected to the circuit board 110 to supply power to the light emitting diodes 120, and an insulating sheet 180 that cuts off the electrical connection between the circuit board 110 and the battery 150.

However, in the related art, when the patch assembly 100 is packaged and is in an unused state, the insulating sheet 180 is simply interposed between the circuit board 110 and the battery 150 in order to prevent the battery 150 from being discharged, and thus there is a problem that the battery 150 is discharged to unnecessarily consume a current while the insulating sheet 180 is easily moved or pulled out by an external force during manufacturing or distribution.

In addition, once the insulating sheet 180 is removed for the use of the patch assembly 100 or inadvertently by a user, even when the use needs to be interrupted, the electrical connection between the circuit board 110 and the battery 150 may not be cut off as before, and thus there is a limit to induce inconvenience in use as well as unnecessary battery waste.

Here, the above-mentioned background art or the related art is intended to be useful for understanding the technical significance of the present invention as information possessed by the present inventor or acquired in the process of deriving the present invention, it is clarified that it does not mean a technology widely known in the technical field to which the present invention belongs before filing of the present invention, and in addition, the drawing reference numerals in the related art have no relation to the drawing reference numerals of the present invention.

### PRIOR ART LITERATURE

### Patent Literature

(Patent Document 1) Korean Laid-open Patent Application No. KR10-1664152 B1 (Oct. 4, 2016)

(Patent Document 2) Korean Laid-open Patent Application No. KR10-1792659 B1 (Oct. 26, 2017)

(Patent Document 3) Korean Laid-open Patent Application No. KR10-2018-0089464 A (Aug. 8, 2018)

(Patent Document 4) Korean Laid-open Patent Application No. KR10-2067101 B1 (Jan. 10, 2020)

(Patent Document 5) Korean Laid-open Patent Application No. KR10-1413560 B1 (Jun. 24, 2014)

(Patent Document 6) Korean Laid-open Patent Application No. KR10-2009-0016249 A (Feb. 13, 2009)

(Patent Document 7) Korean Laid-open Patent Application No. KR10-2015-0135618 A (Dec. 3, 2015)

### SUMMARY

### Technical Problem

Accordingly, the present inventor has an idea of solving technical limitations and problems of a conventional patch-type LED phototherapy apparatus while comprehensively considering the above-mentioned matters, and has devised the present invention as a result of constant research and effort to develop a phototherapy apparatus with a new structure capable of reducing unnecessary battery waste by surely preventing causing a discharge in which a battery unnecessarily consumes a current by electrically separating a circuit board from the battery when the phototherapy apparatus is not in use.

Therefore, a technical problem and objective to be solved by the present invention is to provide an ultra-compact phototherapy apparatus for treating skin diseases, which may prevent the battery from unnecessarily consuming electric power when not in use.

In addition, another technical problem and objective to be solved by the present invention is to provide the ultra-compact phototherapy apparatus for treating skin diseases, which may prevent the circuit board and the battery from being electrically separated during use.

Here, the technical problem and objective to be solved by the present invention are not limited to the technical problems and objectives mentioned above, and other technical problems and objectives not mentioned above may be clearly understood by those skilled in the art from the following descriptions.

### Technical Solution

Specific means according to an aspect of the present invention for achieving the above-mentioned technical objectives and for solving problems or technical problems of the related art provides an ultra-compact phototherapy apparatus for treating skin diseases including a PCB substrate in which one or more light emitting elements and a first metal electrode are formed on an upper surface and a second metal electrode is formed on a lower surface, a battery having a first electrode on an upper surface, which is in contact with the second metal electrode on the lower surface of the PCB substrate, and a second electrode on a lower surface, and an electrode body formed so that the battery is inserted inside and the PCB substrate is inserted in slidingly a front-rear direction, of which an upper inner side is selectively contact with the first metal electrode of the PCB substrate in a state in which a lower inner side is in contact with the second electrode of the battery depending on a sliding insertion position of the PCB substrate to supply or shut off power of the battery to the PCB substrate.

Accordingly, the present invention may surely prevent causing a discharge in which the battery unnecessarily consumes a current by electrically separating a PCB substrate from the battery when the phototherapy apparatus is not in use.

In addition, a preferred aspect of the present invention further includes: a pair of left and right first stoppers formed in front of the first metal electrode in a direction of being inserted into the electrode body of the upper surface of the PCB substrate; and a contact protrusion that is formed on both sides of an upper portion of the electrode body to cause contact interference with the first stopper before sliding insertion of the PCB substrate and is maintained in that state, and maintains a state of being in contact with the first metal electrode while being elastically deformed by force of a certain level or more during the sliding insertion of the PCB substrate, and accordingly, the electrically separated state between the PCB substrate and the battery may be maintained more stably.

In addition, a preferred aspect of the present invention further includes a pair of left and right second stoppers that is formed in rear of the first metal electrode in the direction of being inserted into the electrode body of the upper surface of the PCB substrate to maintain a position so that the contact protrusion does not come off from a state of being in contact with the first metal electrode, and accordingly, it is possible prevents the PCB substrate and the battery from being separated from the electrically connected state, and of course, a user may feel the electrically connected state of the PCB substrate and the battery with a tactile sensation.

Further, in a preferred aspect of the present invention, the first stopper is formed in a shape in which a width gradually narrows upward on both front and rear sides in the direction of being inserted into the electrode body, and the contact protrusion is formed in a shape in which a width gradually narrows downward on both front and rear sides in a direction in which the PCB substrate is inserted, and accordingly, the sliding insertion of the PCB substrate by force of a certain level or more may be performed more smoothly.

In addition, a preferred aspect of the present invention further includes first and second slots formed on both side wall portions of the electrode body at a predetermined distance, and a guide protrusion that is formed protruding on both sides of the PCB substrate to be fitted into the first slot before sliding insertion of the PCB substrate, and is maintained in that state, and maintains a state of being fitted into the second slot by being separated and moved from the first slot while being elastically deformed by force of a certain level or more during the sliding insertion of the PCB substrate, and accordingly, an electrically separated state between the PCB substrate and the battery may be maintained more stably, and of course, it is possible to prevent the PCB substrate and the battery from being naturally separated from an electrically connected state.

Further, in a preferred aspect of the present invention, the guide protrusion is formed in a shape in which a width gradually narrows outward on both front and rear sides in a direction of being inserted into the electrode body, and accordingly, the sliding insertion of the PCB substrate by force of a certain level or more may be performed more smoothly.

In addition, a preferred aspect of the present invention, further includes a guide that is integrally formed at upper ends of left and right side wall portions of the side wall portions formed on four sides of the electrode body to guide sliding insertion of the PCB substrate, and has the contact protrusion formed protruding on a lower surface facing the first metal electrode of the PCB substrate, and accordingly, an operation for the sliding insertion of the PCB substrate may be performed more easily and smoothly.

### Advantageous Effects

In order to solve the above technical problems, according to the technological scope and the embodiment of the present invention on which a specific solution is based, it is possible to surely prevent causing a discharge in which a battery unnecessarily consumes a current by maintaining stably a state in which a PCB substrate and a battery are electrically separated when a phototherapy apparatus is not in use.

In addition, it is possible to prevent the PCB substrate and the battery from being naturally separated from an electrically connected state for the use, and of course, a user may feel the electrically connected state of the PCB substrate and the battery with a tactile sensation, and thus usability may be improved.

Here, the effects of the present invention are not limited to the above-mentioned effects, and other effects not mentioned will be clearly understood by those skilled in the art from the description of the claims.

### BRIEF DESCRIPTION OF THE DRAWINGS

FIG. 1 is a perspective view showing an example of a phototherapy apparatus according to the related art.

FIG. 2 is a perspective view showing an exploded phototherapy apparatus according to an embodiment of the present invention.

FIG. 3 is a perspective view showing a phototherapy apparatus according to an embodiment of the present invention.

FIG. 4 is a front view showing a state in which a PCB substrate and a battery are electrically separated from among components of a phototherapy apparatus according to an embodiment of the present invention

FIG. 5 is a front view showing a state in which a PCB substrate and a battery are electrically connected among components of a phototherapy apparatus according to an embodiment of the present invention

FIG. 6 is a rear perspective view showing a PCB substrate among components of a phototherapy apparatus according to an embodiment of the present invention.

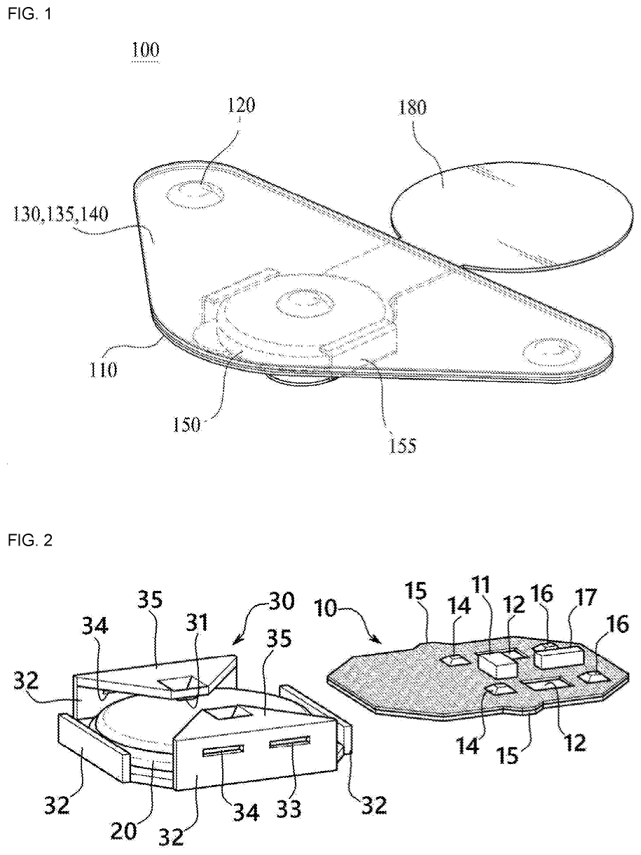

### DETAILED DESCRIPTION OF THE EMBODIMENTS

Hereinafter, embodiments of the present invention will be described in detail with reference to the accompanying drawings.

Prior to this, terms to be described below are terms defined in consideration of functions in the embodiments of the present invention, and clarify that the concept is consistent with the technological scope of the present invention and should be construed as a meaning that is accepted or commonly recognized in the art.

In addition, when it is determined that a detailed description of a well-known function or configuration may unnecessarily obscure the gist of the present disclosure, the detailed description thereof is omitted.

Here, the accompanying drawings are shown by exaggerating or simplifying a part for the convenience and clarity of explanation and understanding of the structure and operation of the technology, and it is clear that each component does not exactly match its actual size and shape.

In addition, in the present specification, the term and/or is meant to include a combination of a plurality of related description items or any item of the plurality of related description items, and when a certain part “includes” a certain component, it means that another component may be further included rather than excluding another component unless otherwise specified.

That is, it means that there are features, numbers, steps, actions, components, parts or combinations thereof as described herein, and it should be understood that it does not preclude the existence or addition of one or more other features or numbers. steps, actions, components, parts or combinations thereof.

In addition, the meanings of the terms “part” and “unit” mean at least one function intended in the system, or a modular form that acts as a unit or plays a role to process a certain operation, and can be implemented by means such as hardware, software, or a combination of hardware and software, or by a device or assembly that can perform independent operations.

In addition, terms such as upper end, lower end, upper surface, lower surface, or upper portion, lower portion, upper side, lower side, front and rear, left and right, etc. are used for convenience to distinguish relative positions in each component. For example, the upper side in the drawing may be named or called as the upper portion and the lower side in the drawing may be named or called as the lower portion, and a longitudinal direction may be named or called as a front-rear direction and a lateral direction may be named or called as a left-right direction.

In addition, terms such as first and second may be used to describe various components. That is, the terms such as first and second may be used to distinguish one component from the another. For example, a first component may be named as a second component as long as it does not deviate from the scope of protection of the present invention, and the second component may also be named as the first component.

Meanwhile, a ultra-compact phototherapy apparatus for treating skin diseases according to an embodiment of the present invention is protected by a sealing treatment with a hydrogel layer or the like that transmits light energy emitted from a light emitting element to the outside to transmit it to the skin, and then, it may be used by adhering to a bent part of the human body using a medical adhesive band.

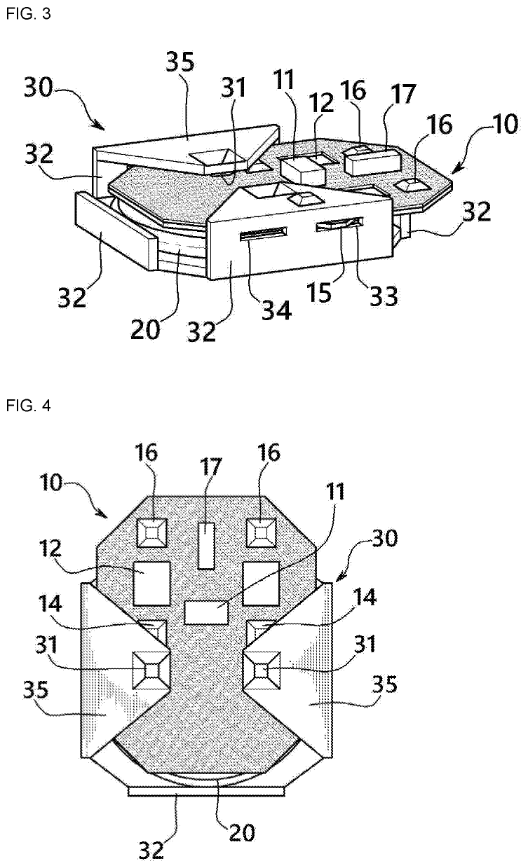

As shown in FIGS. 2 to 6, main components of the phototherapy apparatus according to the embodiment of the present invention include a PCB substrate 10, a battery 20, and an electrode body 30.

The PCB substrate 10 is for electrically connecting a light emitting element 11 and the battery 20, an upper surface of the PCB substrate 10 is covered with an insulating layer, and the PCB substrate 10 may be formed to be slidably inserted into an internal space of the electrode body 30 in a front-rear direction.

That is, the light emitting element 11 that emits light of a specific wavelength, a resistor 17, and the like are mounted on the upper surface of the PCB substrate 10, and a pair of left and right or one of first metal electrodes 12 (positive pole) is formed to be exposed by removing a part of the insulating layer, and a second metal electrode 13 (negative pole) is formed on a lower surface of the PCB substrate 10.

In addition, a pair of left and right first stoppers 14 are formed in front of the first metal electrodes 12 in a direction of being inserted into the electrode body 30 of the upper surface of the PCB substrate 10.

That is, the pair of left and right first stoppers 14 cause contact interference with a contact protrusion 31 of the electrode body 30 before the PCB substrate 10 is inserted into the electrode body 30, and serve to stably maintain an electrically separated state between the PCB substrate 10 and the battery 20.

Here, both front and rear sides of the first stopper 14 are formed in a shape in which a width gradually narrows toward an upper end in a direction of being inserted into the electrode body 30, that is, an inclined surface, and accordingly, an operation for the sliding insertion of the PCB substrate 10 by force of a certain level or more may be performed more smoothly.

In addition, a pair of left and right second stoppers 16 are formed in rear of the first metal electrodes 12 in the direction of being inserted into the electrode body 30 of the upper surface of the PCB substrate 10.

That is, the pair of left and right second stoppers 16 insert the PCB substrate 10 into the electrode body 30 and serve to maintain the position so that the contact protrusion 31 of the electrode body 30 does not come off from a state of being in contact with the first metal electrode 12 of the PCB substrate 10.

Meanwhile, the PCB substrate 10 may be made of a material having flexibility and elastic restoring force characteristics, and may be formed in various sizes in consideration of an area of an affected part.

For example, it is desirable that the PCB substrate 10 is made of a flexible printed circuit board (FPCB) that flexibly bends in response to bending of an adhesion site of the skin in order to facilitate adhesion to the skin, but the embodiment is not limited thereto, and may be composed of a polyethylene terephthalate (PET) substrate, a polyimide substrate, or a polydimethylsiloxane (PDMS) substrate.

In addition, a guide protrusion 15 is formed protruding on both sides of the PCB substrate 10.

That is, the guide protrusion 15 is primarily fitted into a first slot 33 of the electrode body 30 before the PCB substrate 10 is completely slid and inserted into the electrode body 30, and is maintained in that state, and maintains a state of being fitted into a second slot 34 by being separated and moved from the first slot 33 while being elastically deformed by force of a certain level or more during sliding insertion of the PCB substrate 10, and accordingly, an electrically separated state between the PCB substrate 10 and the battery 20 may be maintained more stably and it is possible to prevent the PCB substrate 10 and the battery 20 from being naturally separated from an electrically connected state.

Here, the guide protrusion 15 is formed in an inclined surface in which a width gradually narrows outward on both front and rear sides in the direction of being inserted into the electrode body 30, and accordingly, the sliding insertion of the PCB substrate 10 by force of a certain level or more may be performed more smoothly.

Meanwhile, one or more micro LED chips or laser diodes may be adopted as the light emitting element 11, which emits light in a wavelength band of 300 nm to 1300 nm that has a pain-relieving effect as well as a treatment for whitening, wrinkle improvement, acne, erythema, actinic keratosis, etc. and a skin improvement (or regeneration) effect.

Here, a number of light emitting elements 11 may be selected in various ways in consideration of an area of the PCB substrate 10, a condition of an affected area or the skin, etc., and the wavelength band thereof may be used selectively in various ways depending on a usage mode and the like.

That is, various effects shown for each wavelength band of light rays emitted from the light emitting element 11 are shown in Table 1 below.

TABLE 1

| Way elength | | Action effect and | Utilized |
|---|---|---|---|
| range | Category | Application field | light source |
| UV-C | Far ultraviolet | Bactericidal | LED light |
| | (100-280 nm) | and clean action | source (250 nm) |
| | | Bio-medical sensor | UV Fluorescent |
| | | | lamp |
| UV-B | Medium | Formation of vitamin D | LED (315 nm) |
| | ultraviolet | Leukoplakia, Psoriasis | UV Fluorescent |
| | (280-320 nm) | treatment device | lamp |
| UV-A | Near | Treatment of | LED (365 nm, |
| | ultraviolet | atopic dermatitis | 380 nm) |
| | (320-400 nm) | Scleroderma, | UV Fluorescent |
| | | Treatment of mycosis | lamp |
| Visible light | R,G,B | Treatment of | LED (All |
| | (400-780 nm) | neonatal jaundice | wavelength |
| | | Treatment of acne | band) |
| | | and age spots | |
| | | Skin improvement, | |
| | | Optic nerve treatment | |
| | | Depression, Aesthetic | |
| | | treatment | |
| IR-A | Near infrared | Pain treatment (relief) | LED/LD |
| | (780 nm-2.5 μm) | Promotion of skin | (830 nm) |
| | | regeneration | Halogen lamp |
| | | Promotion of | |
| | | suturing of affected | |
| | | area after surgery | |
| IR-B | Far infrared | Pain treatment | Halogen- |
| | (medicine) | (heat treatment) | tungsten lamp |
| | (2.5 μm-50 μm) | Musculoskeletal | (4-18 μm) |
| | | treatment | |
| IR-C | Far infrared | Nothing known | |
| | (physics) | | |
| | (50 μm-1 mm) | | |

In addition, it is desirable that the light emitting element 11 adopts a chip on board (COB) type in which a chip is connected to the PCB substrate 10 by wire bonding and then molded.

That is, since the light emitting element 11 of the COB type is extremely small, has a good heat dissipation function, and has a minute protrusion, there is little discomfort felt by the user when it adheres to the affected area, and grounding is not required, and thus it is possible to prevent a possibility of accidents due to an electric current that may occur when an electrical product adheres to the skin.

The battery 20 is inserted inside the electrode body 30 so that a first electrode (negative pole) is located on an upper surface and a second electrode (positive pole) is located on a lower surface in order to supply power to the PCB substrate 10. The first electrode (negative pole) on the upper surface thereof is in contact with the second metal electrode (negative pole) 13 on the lower surface of the PCB substrate 10, and the second electrode (positive pole) on the lower surface thereof is in contact with a lower inner side of the electrode body 30.

That is, both electrodes of the battery 20 are in contact with both terminals of the PCB substrate 10 to supply power so that the light emitting element 11 may emit light.

Here, lithium polymer, lithium ion, nickel hydrogen, or the like which may perform wired/wireless charging and has a thin shape may be adopted as the battery 20.

In addition, a battery having a flexible structure known to have flexibility and elastic restoring force characteristics may be adopted as the battery 20.

The electrode body 30 serves as a switch that turns on/off supplying of power of the battery 20 to the light emitting element 11, accommodates the PCB substrate 10 and the battery 20, and is formed in a flat and slim shape so that it may be attached to the human body.

In addition, the electrode body 30 is formed with a space inside which the battery 20 is inserted and mounted so that the user may selectively supply or shut off the power of the battery 20 to the PCB substrate 10, and a side wall portion 32 is formed at four ends thereof so that the PCB substrate 10 may be slidably inserted in the front-rear direction while preventing the battery 20 from moving.

That is, a distance between the front and rear and the left and right side wall portions 32 is slightly larger than a diameter of the battery 20, and particularly a distance between the left and right side wall portions 32 is formed to be slightly larger than a left and right width of the PCB substrate 10.

In addition, the front and rear side wall portions 32 are formed to be slightly lower in height than the left and right side wall portions 32 so that the PCB substrate 10 may be slid-inserted in the front-rear direction.

In addition, the lower inner side of the electrode body 30 is in contact with the second electrode (positive pole) of the battery 20 depending on a sliding insertion position of the PCB substrate 10, and in this state, an upper inner side of the electrode body 30 is formed so as to selectively contact the first metal electrode 12 of the PCB substrate 10.

That is, the contact protrusion 31 is formed at an upper portion of the side wall portions 32 of both the left and right sides of the electrode body 30 to cause contact interference with the first stopper 14 before the sliding insertion of the PCB substrate 10, to be maintained in that state, and to maintain a state of being in contact with the first metal electrode 12 while being elastically deformed by force of a certain level or more during the sliding insertion of the PCB substrate 10.

Here, both the front and rear sides of the contact protrusion 31 are formed in a shape in which a width gradually narrows toward a lower end in a direction in which the PCB substrate 10 is inserted, that is, an inclined surface, and accordingly, the sliding insertion of the PCB substrate 10 by force of a certain level or more may be performed more smoothly.

In addition, the first and second slots 33 and 34 are formed on the side wall portions 32 of both sides of the electrode body 30 at a predetermined distance.

That is, the first and second slots 33 and 34 are formed so that the guide protrusion 15 of the PCB substrate 10 is selectively fitted thereto.

In addition, a guide 35 guiding for the smooth sliding insertion of the PCB substrate 10 is integrally formed at upper ends of the left and right side wall portions of the side wall portions 32 of the electrode body 30.

That is, the contact protrusion 31 is formed protruding on a lower surface of the guide 35 facing the first metal electrode 12.

Meanwhile, the electrode body 30 may be made of a material having flexibility and elastic restoring force characteristics and a thickness. For example, it may be formed including at least one of silver (Ag), aluminum (Al), and copper (Cu) and in a form having a thin thickness, but the embodiment is not limited thereto, and it may be made of various materials that are flexible and have high electrical conductivity.

A main operation and an operating principle of the phototherapy apparatus according to the embodiment of the present invention will be described with reference to FIGS. 4 to 6.

First, before using the phototherapy apparatus, since the PCB substrate 10 is not completely inserted into the electrode body 30 as shown in FIG. 4, it is possible to maintain a state in which the PCB substrate 10 and the battery 20 are electrically separated, and prevent unnecessary power consumption of the battery 20.

At this time, the electrically separated state between the PCB substrate 10 and the battery 20 may be stably maintained by maintaining a state in which the first stopper 14 of the PCB substrate 10 causes contact interference with the contact protrusion 31 of the electrode body 30.

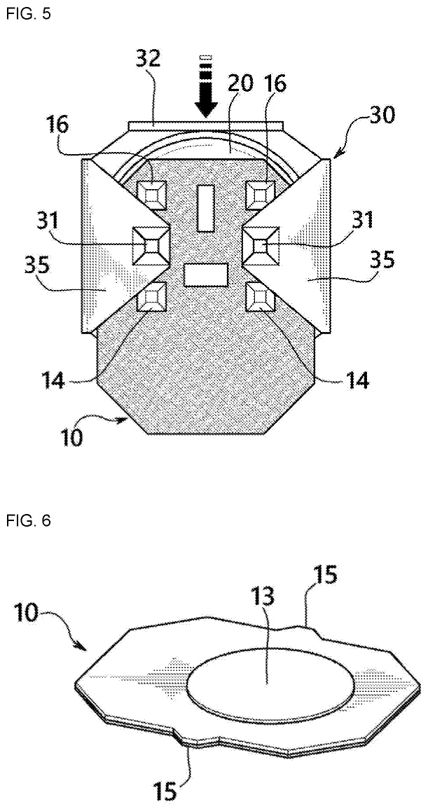

In order to use the phototherapy apparatus in this state, when the PCB substrate 10 is pushed and slid forward to be completely inserted into the electrode body 30 as shown in FIG. 5, the contact protrusion 31 of the electrode body 30 may be maintained in contact with the first metal electrode 12 while being elastically deformed by a pushing force acting above a certain level in this process.

At this time, the first electrode (negative pole) located on the upper surface of the battery 20 is in contact with the second metal electrode 13 on the lower surface of the PCB substrate 10, and the second electrode (positive pole) located on the lower surface of the battery 20 is in contact with the first metal electrode 12 of the PCB substrate 10 via the contact protrusion 31 of the electrode body 30, and thus the power of the battery 20 is supplied to the PCB substrate 10.

That is, when the power of the battery 20 is turned on and off depending on the sliding insertion position of the PCB substrate 10 and when the power is supplied from the battery 20 to the PCB substrate 10, the light emitting element 11 emits light in an optimized wavelength band corresponding to an affected area or a symptom by an operation programmed and set in advance, and accordingly, it is possible to provide an ideal therapeutic effect by attaching to various parts of the skin.

Therefore, when the user does not use the phototherapy apparatus for carrying or storage, unnecessary waste of the battery 20 may be reduced.

Meanwhile, the second stopper 16 of the PCB substrate 10 maintains a position so as not to come off from the state in which the contact protrusion 31 of the electrode body 30 is in contact with the first metal electrode 12, and thus it is possible to prevent the PCB substrate 10 and the battery 20 from being separated from an electrically connected state, and of course, the user may feel the electrically connected state of the PCB substrate 10 and the battery 20 with a tactile sensation, and thus usability may be improved.

Moreover, before using the phototherapy apparatus, that is, before sliding insertion of the PCB substrate 10 into the electrode body 30, the guide protrusion 15 is maintained in a state of being fitted into the first slot 33 of the electrode body 30, and thus, the electrically separated state between the PCB substrate 10 and the battery 20 may be maintained more stably.

Further, in order to use the phototherapy apparatus, when the PCB substrate 10 is pushed and slid forward to be completely inserted into the electrode body 30, the guide protrusion 15 of the PCB substrate 10 maintains a state of being fitted into the second slot 34 by being separated and moved from the first slot 33 while being elastically deformed by a pushing force acting above a certain level in this process, and thus it is possible to more surely prevent the PCB substrate 10 and the battery 20 from being naturally separated from an electrically connected state, and additionally, the user may feel the electrically connected state of the PCB substrate 10 and the battery 20 with a tactile sensation, that is, a click feeling, and thus usability may be improved.

Furthermore, since the entire lower surface of the PCB substrate 10 is in surface contact with the upper surface of the battery 20, and the entire lower surface of the battery 20 is in surface contact with the inside surface of a lower portion of the electrode body 30, heat generated from the light emitting element 11 is dissipated very efficiently through the electrode body 30 via the PCB substrate 10 and the battery 20, thereby preventing a risk of low-temperature burns caused by overheating of the light emitting element 11 located close to the skin.

Meanwhile, the present invention is not limited by the above-described embodiments and the accompanying drawings, but may be variously modified and applied into unexemplified various forms within a scope not departing from the technical spirit of the present invention, and of course, it is clear to those skilled in the art to which the present invention pertains that the replacement of each component and the modification to other equivalent embodiments may be widely applied.

Therefore, the content relating to the modification and application of the technical features of the present invention should be interpreted as being included within the spirit and scope of the present invention.

DESCRIPTION OF
REFERENCE NUMERALS
10: PCB substrate
11: Light emitting element
12: First metal electrode
13: Second metal electrode
14: First stopper
15: Guide protrusion
16: Second stopper
17: Resistor
20: Battery
30: Electrode body
31: Contact protrusion
32: Side wall portion
33: First slot
34: Second slot
35: Guide

What is claimed is:

1. An ultra-compact phototherapy apparatus for treating skin diseases, the apparatus comprising:
a PCB substrate in which one or more light emitting elements and a first metal electrode are formed on an upper surface and a second metal electrode is formed on a lower surface;
a battery having a first electrode on an upper surface, which is in contact with the second metal electrode on the lower surface of the PCB substrate, and a second electrode on a lower surface; and
an electrode body formed so that the battery is inserted inside and the PCB substrate is inserted in slidingly a front-rear direction, of which an upper inner side is selectively in contact with the first metal electrode of the PCB substrate in a state in which a lower inner side is in contact with the second electrode of the battery depending on a sliding insertion position of the PCB substrate to supply or shut off power of the battery to the PCB substrate.

2. The apparatus of claim 1, further comprising:
a first stopper formed in front of the first metal electrode in a direction of being inserted into the electrode body of the upper surface of the PCB substrate; and
a contact protrusion that is formed on both sides of an upper portion of the electrode body to cause contact interference with the first stopper before sliding insertion of the PCB substrate and is maintained in that state, and maintains a state of being in contact with the first metal electrode while being elastically deformed by force of a certain level or more during the sliding insertion of the PCB substrate.

3. The apparatus of claim 2, further comprising
a second stopper formed in rear of the first metal electrode in the direction of being inserted into the electrode body of the upper surface of the PCB substrate to maintain a position so that the contact protrusion does not come off from a state of being in contact with the first metal electrode.

4. The apparatus of claim 2, wherein the first stopper is formed in a shape in which a width gradually narrows upward on both front and rear sides in the direction of being inserted into the electrode body, and
the contact protrusion is formed in a shape in which a width gradually narrows downward on both front and rear sides in a direction in which the PCB substrate is inserted.

5. The apparatus of claim 1, further comprising:
first and second slots formed on both side wall portions of the electrode body at a predetermined distance; and
a guide protrusion that is formed protruding on both sides of the PCB substrate to be fitted into the first slot before sliding insertion of the PCB substrate, and is maintained in that state, and maintains a state of being fitted into the second slot by being separated and moved from the first slot while being elastically deformed by force of a certain level or more during the sliding insertion of the PCB substrate.

6. The apparatus of claim 5, wherein the guide protrusion is formed in a shape in which a width gradually narrows outward on both front and rear sides in a direction of being inserted into the electrode body.

7. The apparatus of claim 2, further comprising
a guide that is integrally formed at upper ends of left and right side wall portions of the side wall portions formed on four sides of the electrode body to guide sliding insertion of the PCB substrate, and has the contact protrusion formed protruding on a lower surface facing the first metal electrode of the PCB substrate.
